(19) United States
(12) Patent Application Publication
(10) Pub. No.: US 2022/0102004 A1
(43) Pub. Date: Mar. 31, 2022

(54) PATIENT MONITORING, REPORTING AND TRACKING SYSTEM

(71) Applicant: Susan L. Abend, Bradenton, FL (US)

(72) Inventor: Susan L. Abend, Bradenton, FL (US)

(21) Appl. No.: 17/487,612

(22) Filed: Sep. 28, 2021

Related U.S. Application Data
(60) Provisional application No. 63/084,249, filed on Sep. 28, 2020.

(51) Int. Cl. G16H 50/20; G16H 10/20; G16H 20/70

(52) U.S. Cl. CPC G16H 50/20; G16H 20/70; G16H 10/20

(57) ABSTRACT

A patient wellbeing and symptom tracking system receives responses from healthcare workers for specific symptoms, occurrences or benchmarks encountered by the patient over execution of normal activities of daily life (ADLs). Different healthcare workers, direct caregivers or administrators may encounter the symptoms at different times, and the full collective body of symptoms can tend to elude detection because of disparate occurrences spread across multiple observers. While individually any one of the gathered symptoms may be incidental and not necessarily warrant an intervention or notification, certain collective symptoms may point to more substantial occurrences or problems deserving of further attention. A series of medical logic expressions denotes particular groups of symptoms and/or severity/symptom qualifications that collectively denote a more serious condition, and prompt direct caregivers and other stakeholders responsible for the health and safety of an individual with IDD to obtain medical consultation.

### RELATED APPLICATIONS

This patent application claims the benefit under 35 U.S.C. § 119(e) of U.S. Provisional Patent App. No. 63/084,249, filed Sep. 28, 2020, entitled “PATIENT MONITORING, REPORTING AND TRACKING SYSTEM,” incorporated herein by reference in entirety.

### BACKGROUND

As medical and healthcare capabilities enlarge with expanding technology, available treatment and longevity options continue to become increasingly available. Healthcare services therefore move towards a compartmentalized approach with different healthcare workers performing more specialized tasks. Care of a particular patient, therefore, involves multiple caretakers at different times. A single caretaker entity may not be in continual observation of a patient for an extended interval, and therefore no single individual observes different symptoms or events that might suggest a trend. In the case of communicative compromise, or an inability of the patient to directly convey symptoms of wellbeing, trends or patterns in patients having developmental and intellectual disabilities (D/ID) or other intellectual disabilities such as dementia can be overlooked.

### SUMMARY

A patient wellbeing and symptom tracking system receives responses from healthcare workers for specific symptoms, occurrences or benchmarks encountered by the patient over execution of normal activities of daily life (ADLs). While individually any one of the gathered symptoms may be incidental and not necessarily warrant an intervention or notification, certain collective symptoms may point to more substantial occurrences or problems deserving of further attention. A series of medical logic expressions denotes particular groups of symptoms and/or severity/symptom qualifications that collectively denote a more serious condition, and define a provider inquiry that should be made to assess further treatment or remedial measures. Different healthcare workers or administrators may encounter the symptoms at different times, and the full collective body of symptoms can tend to elude detection because of disparate occurrences spread across multiple observers. Gathering and coalescing the collective symptoms observed in a single patient triggers a medical logic expression that recognizes the collective symptoms as indicative of a larger potential problem, and renders a provider inquiry for undertaking to investigate and remedy any looming problem.

Configurations herein are directed to health and healthcare delivery disparities in individuals with cognitive disabilities who are unable to communicate specific health history or symptoms to caregivers or healthcare providers. The disclosed approach is based, in part, on the observations that medical treatment needs and direction are often based on direct communication with the patient for reported symptoms and related occurrences or activities. Unfortunately, patients with developmental and intellectual disabilities (D/ID) may be compromised in their ability to convey symptoms and occurrences, such as details about ADLs (Activities of Daily Living), to a caretaker or administrator. Accordingly, configurations herein substantially overcome these shortcomings by providing a system and database for receiving information and observations from caretakers about patient symptoms, storing these symptoms, and matching collective symptoms with medical logic expressions based on collective symptoms and corresponding outcomes, and rendering a provider inquiry for investigation and follow-up to remediate an issue before exacerbation. Symptoms reported from multiple, different providers are therefore coalesced to identify a common medical problem or issue denoted by the collective symptoms from different caretakers, and a medical logic expression recognizing the collective symptoms renders a recommended provider inquiry to be made or next steps to be taken.

In a particular configuration, a network communication and reporting device or server detects and tracks health anomalies in patients having developmental and intellectual disabilities (D/ID) or dementia. An interface receives responses to a sequence of questions in a patient encounter, such that each question pertains to a symptom based on an aspect of health and wellbeing known to be compromised in a patient having a self expression degradation. A storage repository in an EHR provider stores a received response for each symptom. An execution engine coalesces the received responses with a set of conditions for determining a match between the received responses and combinations of symptoms indicative of a provider recommendation, typically via a script of the medical logic for denoting the condition. An output device renders the matched provider recommendation through one of several reports.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a patient date environment suitable for use with configurations herein;

FIG. 2 shows a data flow of patient data employed in computing the provider recommendation of FIG. 1;

FIG. 3 shows an example of medical logic and recommendations as computed in FIG. 2.

FIG. 4 shows an example of database tables invoked in the data flow of FIG. 3;

FIG. 5 is an example of symptom values used in the patient symptom of FIG. 4;

FIG. 6 is an example of medical logic entries used in the medical logic table of FIG. 4;

FIG. 7 shows a system view of integration of an EHR platform with the medical logic as encapsulated in the table of FIG. 6;

FIG. 8 is a flowchart of patient diagnosis using the gathered symptoms and a medical logic expression as in FIG. 7;

FIG. 9 is an example of a patient summary report for a healthcare professional generated as in FIG. 2;

FIG. 10 is an example of a patient risk report for a caregiver generated as in FIG. 2; and

FIG. 11 is a population health quality report generated as in FIG. 2.

### DETAILED DESCRIPTION

In the configurations discussed below, several terms are frequently used.

A patient is generally a medical care recipient having D/ID, and therefore has limitations in communication and reporting of medical conditions, requiring observation and/or testing for assessment of many or all physiologic conditions.

An Electronic Healthcare Record is a storage medium for patient medical records in accordance with applicable privacy and legal requirements such as HIPAA (Health Insurance Portability and Accountability Act), and handles access control for multiple healthcare provider entities to guard against unauthorized access.

A symptom is an observed or examined condition of a patient defined by a value, either Boolean, numeric or enumerated.

A condition is a conclusion of a medical state or outcome based on values of one or more symptoms, often associated with a recommended treatment or course of remedial action.

Medical logic encapsulates medical expertise as a logical sequence or medical logic expression for defining conditions leading to a diagnosis. The medical logic is defined as a logical expression in a syntax based on a script, typically recognized by an EHR system. The syntax allows references to general electronic health records having procedures, medication, and physiology such as medications, height, gender (sex) and weight. A medical logic expression is an expression that may be parsed by the EHR system for evaluation, sometimes referred to as an algorithm for a particular condition.

A provider recommendation is an output regarding a particular patient based on a set of symptoms, and may be a likely diagnosis of an ailment or state of the patient or may indicate a direction, task or action to obtain a diagnosis.

The approach outlined herein depicts a storage and analysis system to implement a method for detecting and tracking health anomalies in patients having developmental and intellectual disabilities (D/ID) by rendering an interface having a sequence of questions, such that each question pertains to a symptom, and each symptom is based on an aspect of health and wellbeing known to be compromised in a patient having D/ID. The system receives a response for each symptom, and coalesces the received responses with a set of conditions for determining a match between the received responses and combinations of symptoms indicative of a provider inquiry. In patients with D/ID, it can be problematic to identify groups of symptoms over time which, when taken in aggregate, may point to a more serious condition. Otherwise, the individual symptoms may merely be observed by different caretakers/healthcare providers at different times, giving rise to a scenario where no one can “connect the dots” and appreciate a possible condition pointed to by collective consideration of multiple, individually inconsequential symptoms. The system then renders the matched provider inquiry or action on an output device for further follow up by the provider.

An input module presents a sequence of questions that may be further subdivided into cognizance areas, such that each cognizance area defines a grouping of symptoms pertaining to a common health topic. For example, the cognizance areas may include ADLs (Activities of Daily Living), observations, ambulation, communications, eating, elimination, environment, negative occurrences, stress, sleep, physiologic data from wearable sensor devices, substances, medications, and health problem list. Such information may be difficult to ascertain from a non-communicative patient. Observed signs/symptoms made by another, such as the previous shift, may be difficult to derive a troublesome pattern from.

The database includes a series of tables including an ordering of each of the symptoms according to a symptom name and range of applicable values for storage as responses to the sign/symptom. Each sign/symptom gathered from the GUI receives a range or value as appropriate, such as a simple Yes/No, a numerical range, or other value.

The database also includes, for each of the conditions, a medical logic expression for identifying a set of signs/symptoms based on the likelihood that the identified symptoms are deterministic of the condition. In other words, each medical logic expression identifies a collective set of symptoms that may be indicative of a condition and corresponding remedial action based on a role specific designation of tasks and/or information. In general, different roles within the circle of care of the patient receive a recommendation specific to that role. Healthcare providers having a level of medical expertise incur a different role or response action than an immediate caretaker or family member, for example. The result is a notifications for each responsible care member (direct caregiver, case manager, physician etc) that is tailored to their role in managing health and safety.

In operation with a received set of signs/symptoms, coalescing includes assigning a condition index based on the received responses to the signs/symptoms, and storing the received responses in a repository (database). Invoking the medical logic expressions and signs/symptoms, the system identifies the condition index based on a match defined in a medical logic expression, retrieves the provider inquiry and renders the provider inquiry. The provider inquiry is likely rendered/received by a different healthcare provider upon a successive visit or examination, as the signs/symptoms may cover an extended timeframe, and is generally indicative of a successive action to be taken for further remediation or examination.

The system described includes a data input module, processing system and reporting module for denoting well described health and healthcare delivery disparities in individuals with cognitive disabilities who are unable to communicate specific health history or symptoms to caregivers or healthcare providers.

The input module accepts specific data regarding observable behaviors, observable biological signals, and answers to questions asked of individuals with cognitive disabilities, healthcare history and current medications. Information can be input into the system in a data entry interface, or by electronic transfer of physiologic data from wearable device vendors, electronic health records, or from the Center for Medicare and Medicaid Services, for example. Inputs include both subjective data such as symptoms and objective data based on signs and direct observation. Current and past medications are also relevant. Input module data is transferred to a database containing specific medical logic expressions designed to detect health issues from that data. Medical logic expressions identify genetic syndrome-specific health issues in Prader Willi syndrome, Down Syndrome, Angleman syndrome, Fragile X syndrome, Smith-Magenis syndrome, Smith-Lemli-Opitz syndrome, Noonan syndrome, myotonic dystrophy, tuberous sclerosis, Rett syndrome, William's Syndrome, cerebral palsy, and genetic syndromes not otherwise specified, as well as health issues that occur in those with intellectual and developmental disabilities, autism, traumatic brain injuries and dementia. Other intellectual/communication abnormalities and IDDs may be encompassed as well. These medical logic expressions are linked to case statements reportable about a respective patient. The case statements are generated within 4 types of reports that prompt and teach caregivers, advocates, healthcare providers to provide necessary treatment and/or preventative care for each individual that meets each medical logic expression's criteria:

1) a report prompting caregivers, guardians and/or advocates to contact health providers on behalf of the individual with cognitive disability for specific issues identified by the system's medical logic expressions,

2) a report prompting caregivers, guardians and/or advocates to contact health providers to assess or reassess unresolved issues detected in previous reports

3) a report containing evidence of changes in health and function in each individual from the previous data input encounter that prompt all healthcare providers and

4) a report indicating populations that meet criteria for health care deficiencies, (available to defined populations such as states, regions, and residents of support-providing entities.) Reports and data input encounters are available from a web interface, are printable, and can be transferred electronically to an individual's electronic health record.

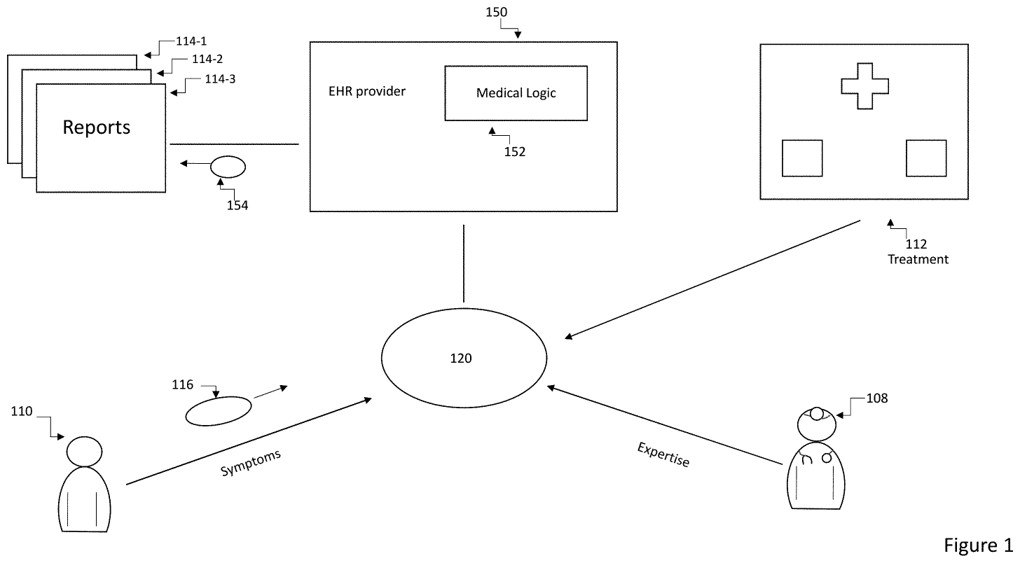

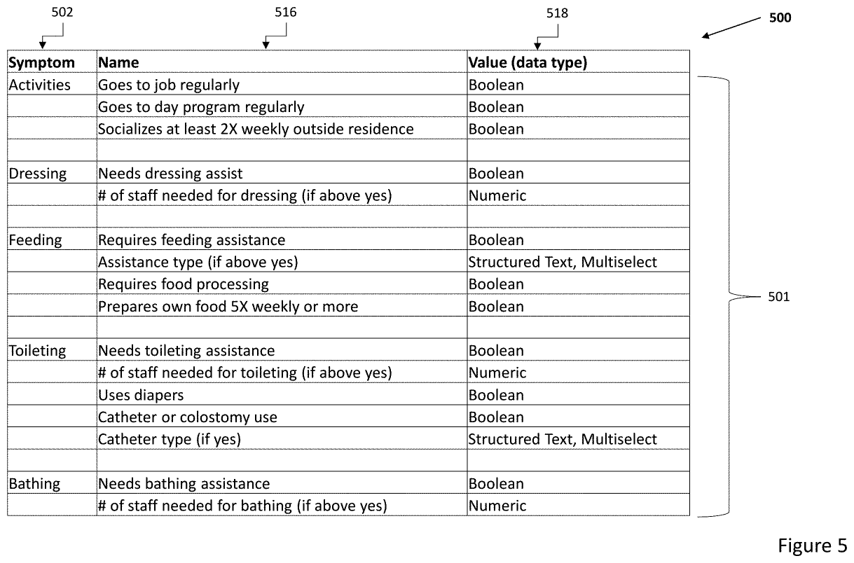

FIG. 1 is a context diagram of a patient data environment 100 suitable for use with configurations herein. In the patient data environment 100, a method for detecting and tracking health anomalies in patients 110 having developmental and intellectual disabilities (D/ID) or dementia includes gathering symptoms 116 from a patient in the form of a patient encounter. The patient encounter addresses a list of symptoms and records values for each, shown further below in FIG. 5. In contrast to conventional approaches, where verbal exchanges between the patient and the healthcare provider define the symptoms, in D/ID patients these often be observed or inferred.

The symptoms 116 and values of each patient are transmitted via a public access network 120 to an Electronic Healthcare Records (EHR) entity 150, typically on a fee-for-services basis. An EHR entity 150 stores confidential patient data and handles access privileges to ensure patient privacy, particularly in view of HIPPA requirements. An EHR service is beneficial because it may be accessed remotely by multiple entities concerned with a patient's health. Particularly in the case of D/ID patients, it can be problematic to recreate or refresh relevant aspects of a patient's medical records and history if EHR access is not provided.

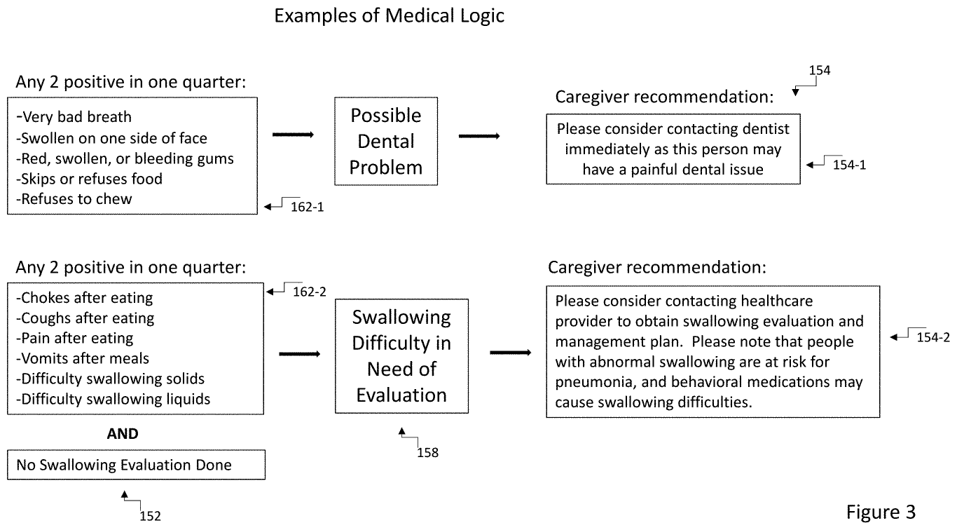

The EHR also stores the full medical history from hospitals 112 and other care facilities, providing a single source repository to the patient's health. Other types of records which may be accessed via the EHR include prescription medications, past medical procedures, and physiological data such as blood chemistry, lab work and physical characteristics, e.g. height, weight, gender. The disclosed approach also generates reports using the EHR data. These reports include provider recommendations as generated herein, and take several different forms depending on the intended reader and the type of report. A healthcare provider report 114-1 (114 generally) contains information for doctors, nurses and healthcare professionals. A caretaker report 114-2 is generated for family members and day-to-day service providers who may not be versed in technical medical aspects. A policy/trends report 114-3 provides statistical data spanning groups rather than individual patients. Various arrangements of report content and expected audience are available, discussed further in FIG. 3, below.

Medical logic 152, gathered from doctors 108 and other medical professionals and experts, takes the form of logic expressions codified as scripts which may be executed in view of the symptoms.

In a typical scenario, the symptoms attributed to the patient via the patient encounter may be invoked by medical logic 152, stored at the EHR 150. The medical logic coalesces the received responses with a set of symptoms for determining a match between the received responses and combinations of symptoms indicative of a provider recommendation. The reports 114 render the matched provider recommendation 154 on an output device for providing or assisting with a diagnoses and treatment plan for the patient.

In contrast to conventional approaches, the medical logic 152 encapsulates sets of symptoms that tend to indicate a particular condition or ailment when occurring together. Personnel turnover for medical caretakers, combined with a patient inability to self-report symptoms, can cause a loss of context where providers are not familiar with the patient. The EHR storage of symptoms, combined with medical logic 152 for coalescing, interpreting, and computing provider recommendations based on the combinations of symptoms, provides a robust approach for D/D patients to ensure critical patient data is not overlooked during treatment and care.

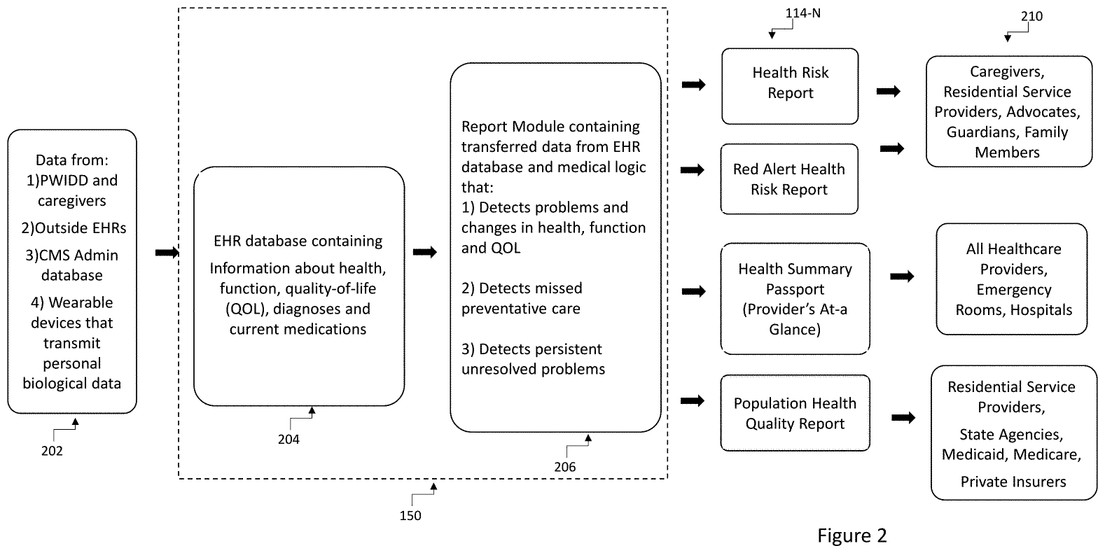

FIG. 2 shows a data flow of patient data employed in computing the provider recommendation of FIG. 1. Data for computing the conditions resulting in a provider recommendation 154 is available from a variety of sources 202, in addition to the reported and gathered symptoms 116. The EHR 150 includes a database 204 for storing the gathered data, and provides a report module 206 for generating the reports 114. A variety of reports 114-N may be generated, and these may be directed of focused on several different audiences 210, including those often referred to as the patient's “circle of care”—the group collectively responsible for patient wellbeing.

Report generation by the report module 206 includes identifying a type of a report for generation based on a healthcare role of a requestor of the report, i.e. doctor, family member, etc. The computed condition is then mapped to a text statement including the provider recommendation based on the type of the report, and the report writer 204 rendering a report 114 including the mapped provider recommendation in conjunction with rendering the condition.

FIG. 3 shows an example of medical logic and recommendations as computed in FIG. 2. Referring to FIGS. 1-3, the medical logic 152 is defined by a plurality of instances 162-1 . . . 162-2 (162 generally), each having a logical statement or equation about a condition. Satisfaction of the symptoms in the instance 162, generally meaning an evaluation of “TRUE,” indicates the existence or diagnosis of the condition.

If the instance evaluates to TRUE, the corresponding condition 158 is established. Each condition maps to a corresponding provider recommendation 154, meaning an affirmative diagnosis and/or statement for remedial treatment or action.

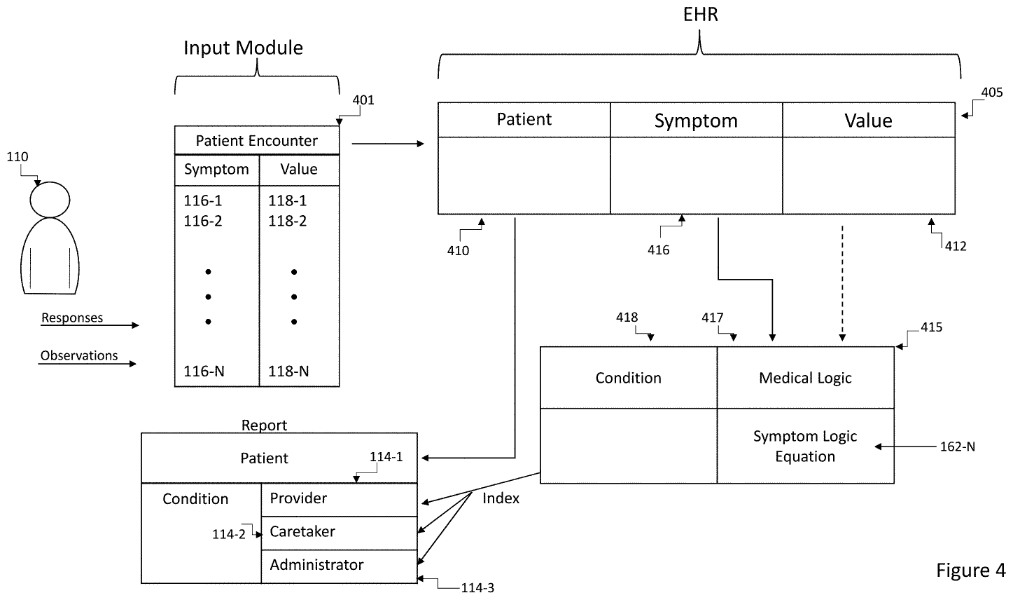

FIG. 4 shows an example of database tables invoked in the data flow of FIG. 3. The symptoms of the patient 110 are based on a list of questions on a form, and responses provided by the healthcare worker based on observations or inference, if the patient cannot verbalize. The sequence of questions are further subdivided into cognizance areas, each cognizance area define a grouping of symptoms pertaining to a common health topic. Referring to FIGS. 1-4, a patient encounter form 401 records symptoms 116 from a healthcare administrator (caretaker, nurse, etc.) The form 401 may be an electronic form on a tablet or laptop, or may be a hardcopy, handwritten form for subsequent data entry. Based on responses and observations by the administrator, symptoms 116 and corresponding values 118-N are recorded. A patient symptom table 405 records the symptoms and values in columns 416 and 412, respectively, indexed by patient column 410.

From the symptoms of a given patient, a condition is ascertained using a medical logic table 415 for identifying a set of symptoms in a medical logic expression based on the likelihood that the identified conditions are deterministic of the condition. The medical logic table 415 has entries in medical logic column 417 and corresponding conditions 419. Generally, each entry has a logic instance 162 referencing one or more symptoms, as well as other data available in the database 204. For each patient, a condition is established by evaluating the logic instance 162 based on the value 412 of the symptoms referenced in the logic instance. If the logic instance evaluates positively, the patient is designated as manifesting the corresponding condition 418, and a report 114 generated based on the condition and patient symptoms and other relevant data, described further in the example reports below. The condition 418 may also indicate a condition index, which maps to a text description of the condition for report rendering. generated report 114 N is a role specific rendering having a different provider recommendation based on the role to which it is directed. In the example of FIG. 1, the provider recommendations vary based on a provider role of healthcare provider 114-1, caretaker 114-2, and administrator/support system roles such as a case manager overseeing a range of related or similar cases. Thus for a given condition, the condition index varies based on the type or report sought to deliver a role specific provider recommendation.

FIG. 5 is an example of symptom values used in the patient symptom table of FIG. 4. Referring to FIGS. 4 and 5, the symptoms 416 and values 412 are based on entries 501 from patient symptom table 500. This table orders each of the symptoms 116 according to a symptom name and range of applicable values 118 for storage as responses to the symptom, and is an example of symptoms 516 and a range of values 512 the symptom can receive based on the patient encounter form 401. The symptoms are also arranged according to cognizance areas 502, discussed above, which simply groups related symptoms together.

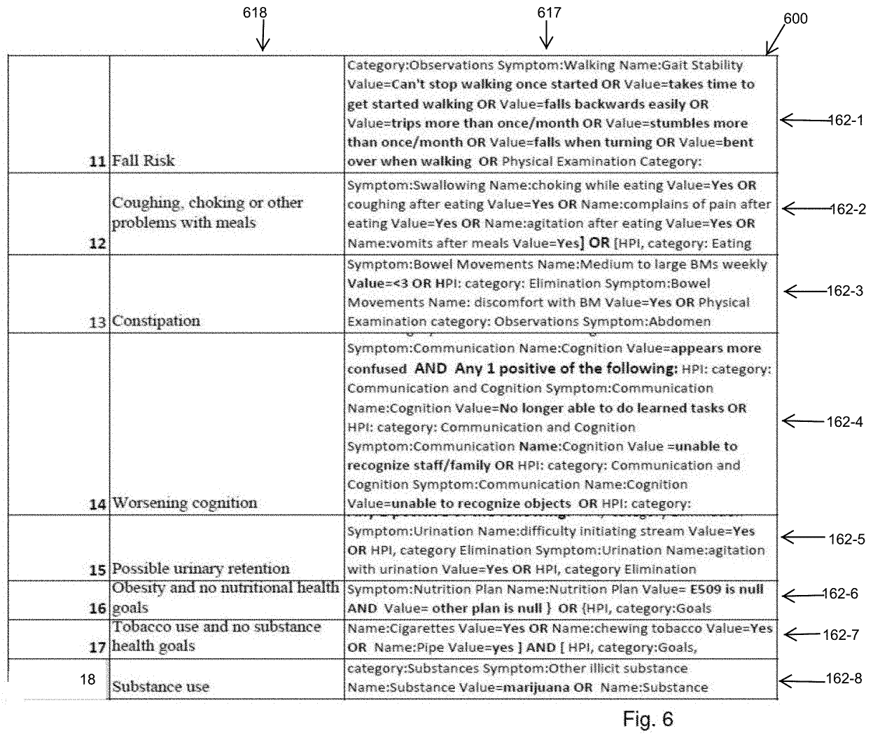

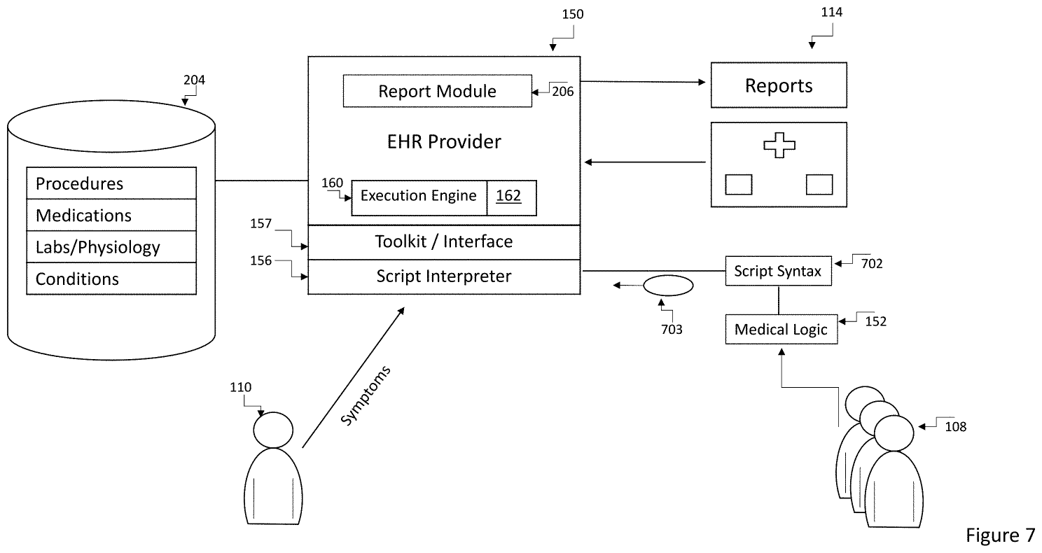

FIG. 6 is an example of medical logic entries used in the medical logic table of FIG. 4. In the table 600 of FIG. 6, each entry in column 617 includes a medical logic instance 162-N for determining or diagnosing a corresponding condition 618 FIG. 7 shows a system view of integration of an EHR platform with the medical logic as encapsulated in the table of FIG. 6. Referring to FIGS. 1-7, the EHR platform 150 includes an execution engine 160 responsive to the medical logic instances 162. It should be noted that the EHR is not limited to a single discrete location, but is likely a distributed system with general network access (i.e. Internet) and hosted by a so-called “cloud” computing service, which generally makes the actual implementation transparent to the user. The significance is that access may be provided anywhere an Internet connection and suitable rendering device are available, subject again to the privacy and privilege assurances implemented by the EHR.

Of particular note in FIG. 7 is an ability to execute the medical logic instances 162 based on the gathered symptoms and values for computing a condition and provider recommendation. The medical logic 152 is gathered from doctors and related experts knowledgeable about particular conditions or symptoms. For a particular condition, a logic expression can codify a combination of symptoms that give rise to that condition. When expressed as a logic equation, a medical logic instance is defined in a script syntax 702 that is interpreted by the EHR 150. The EHR 150 has a script interpreter 156 that parses according to the script syntax, and a toolkit or interface 157 that permits access to the database 204 for retrieving the symptoms 416, values 412 and other patient data relevant to the medical logic. For example, a particular medical logic instance 162 may inquire about certain medications in conjunction with the symptoms. Upon executing one or more logic instances 162, the execution engine 160 invokes the report module 206 for generating relevant reports 114.

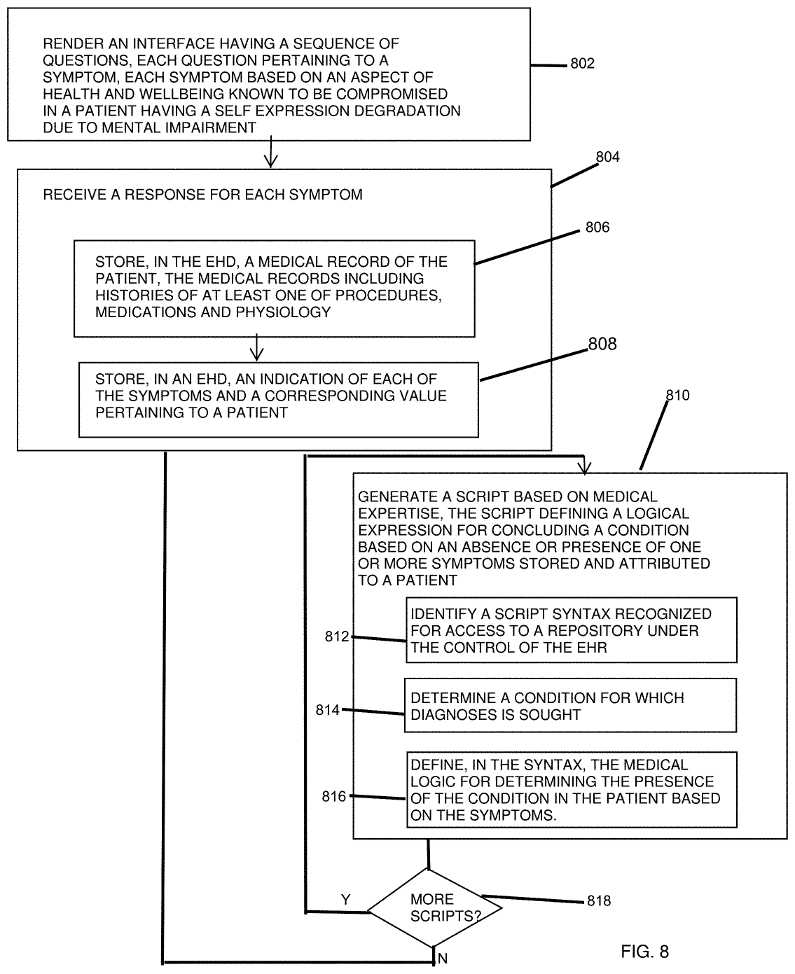

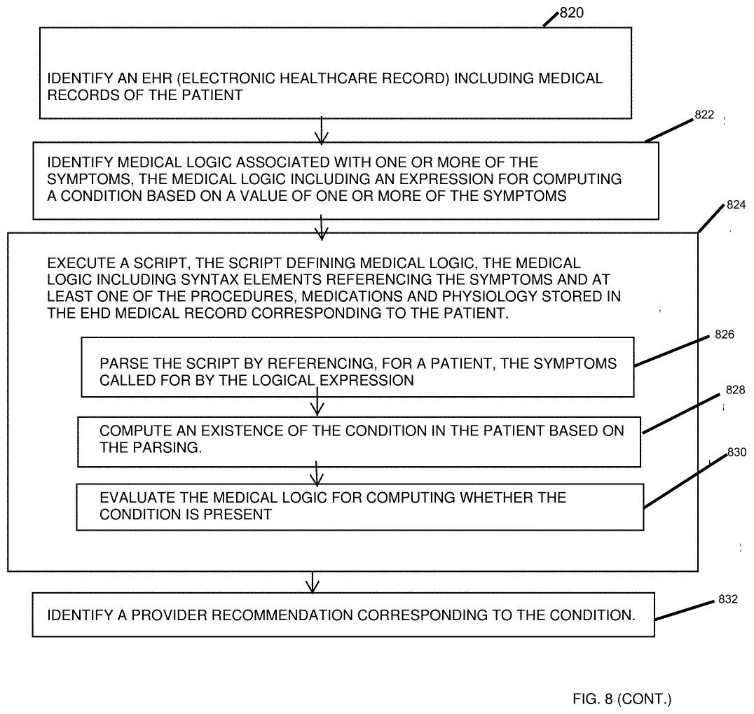

FIG. 8 is a flowchart of patient diagnosis using the gathered symptoms and a medical logic expression as in FIG. 7. Referring to FIGS. 6-8, FIG. 8 depict two independent processes that occur independently—the accumulation of patient symptoms 116 into the EHR database by caretakers, direct service providers and others via the encounter form, and the generation of medical logic 152 for treating the symptoms, gathered from medical staff (doctors) who may be transitioning in and out of various roles. The medical logic encapsulates the medical expertise as a logic statement based on the symptoms for universal application to the symptoms provided. Codification of the medical logic 152 in a script supported by the EHR ensures a consistent body of knowledge is applied to this noncommunicative/limited communicative group of patients within the sphere of medical records privacy afforded by the EHR. Of particular note, often caregivers and direct service providers tend to turnover frequently and may be poorly trained. Observations are gathered without interpretation or judgement. The encounter form 401 is one way of providing objective responses that require little to no interpretation, to maintain consistency in the breadth of responses received. Validation may also be provided by cross-checked responses that look for omissions, inconstancies, or mutual exclusivity in encounter form responses to assess suspect data entry.

Gathering of patient symptoms 116 commences at step 802 by rendering an interface having a sequence of questions, such that each question pertains to a symptom, and each symptom based on an aspect of health and wellbeing known to be compromised in a patient having a self expression degradation due to mental impairment. The patient encounter form 401 or a similar vehicle may be employed, which builds a medical history in the patient symptom table 405. Typically the patient encounter occurs periodically with each patient to ensure changing symptoms are analyzed.

The healthcare administrator receives a response for each symptom on the encounter form 401, as depicted at step 804, This further includes storing, in the EHR database 204, a medical record of the patient, such that the medical records include histories of at least one of procedures, medications and physiology, as disclosed at step 806. The symptom table 405 in the EHR 150 also stores an indication of each of the symptoms and a corresponding value pertaining to a patient, as depicted at step 808.

As an ongoing, current process, the medical logic 152 is accumulated. The script syntax 702 is invoked to generate a script 703 based on medical expertise, such that the script defines a logical expression for concluding a condition based on an absence or presence of one or more symptoms stored and attributed to a patient, as commenced at step 810. The script 703 is an executable or interpretable version of a medical logic instance 162 for a particular condition 518. This includes, at step 812, identifying the script syntax 702 recognized for access to a repository under the control of the EHR 150. The EHR 150 typically has a predetermined script language, or syntax, that executable entities need adhere to in order to be parsed and interpreted correctly in order for the script to access the database 204. The syntax is a set of grammatical rules for expressing the logic, and often can vary slightly in implementation between different EHRs, but generally provide similar logic functions.

The execution engine 160 determines a condition for which diagnoses is sought, as shown at step 814, meaning a condition which a medical logic instance 162 is directed to, as shown at step 814.

The EHR defines, in the syntax, the medical logic for determining the presence of the condition in the patient based on the symptoms by storing the medical logic instance 162 in an entry with the corresponding condition 418 in the database 204 in table 405. Script generation iterates, based on the check at step 818, to generate a library of scripts 703 for medical logic instances 162

Once there are patient symptoms and medical logic instances 162 responsive to those symptoms, patient reports may be generated. A request for a report 114 on a patient 110 includes identifying an EHR (Electronic Healthcare Record) including medical records of the patient, as depicted at step 820. The execution engine 160 identifies medical logic associated with one or more of the symptoms, such that the medical logic includes an expression for computing a condition based on a value of one or more of the symptoms, as depicted at step 822. Each of the medical logic instances 162 codifies the medical logic according to the syntax 702 of the EHR 150. This may occur by merely iterating through known conditions which may be applicable based on one or more of the symptom values. Patients are typically reevaluated for conditions upon entry of a new encounter form 401, which may indicate new symptoms. Patients may be evaluated more frequently, or evaluated against certain medical logic instances, if so requested.

The execution engine 160 executes the identified script, such that the script defines medical logic including syntax elements referencing the symptoms and at least one of the procedures, medications and physiology stored in the EHR medical record corresponding to the patient, as depicted at step 824. In sum, any available data in the database 204, in addition to the symptoms 116 gathered via patient encounters, may be referenced by the script. This includes parsing the script by referencing, for a patient, the symptoms called for by the logical expression defined by the script, as depicted at step 826, computing an existence of the condition in the patient based on parsing, as shown at step 828. The execution engine 160 evaluates the medical logic 152, by executing a script form of a medical logic instance 162, for computing whether the condition is present or established, as depicted at step 830. From the condition, the report module 206 identifies a provider recommendation corresponding to the condition, as shown at step 832.

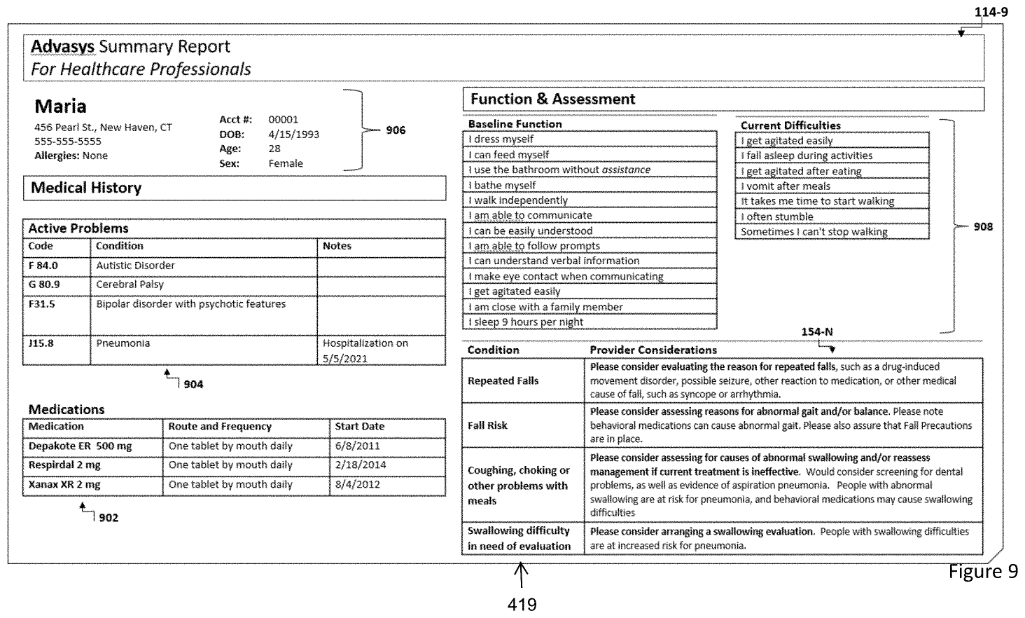

FIG. 9 is an example of a patient summary report for a healthcare professional generated as in FIG. 2. FIG. 9 shows one of several reports that may render the provider recommendation computed by the script. In FIG. 9, a patent summary report 114-9 is directed to healthcare professionals. A number of computed conditions 419-N are rendered, along with corresponding provider recommendations 154-N. Other aspects of the report include medications 902, and previous diagnoses and procedures 904 are also shown. Patient personal information 906 is shown, which is a further advantage of access to the EHR database 204. Other assessments 908 reflect recent assessments pertinent to the case to be conveyed to the healthcare professional.

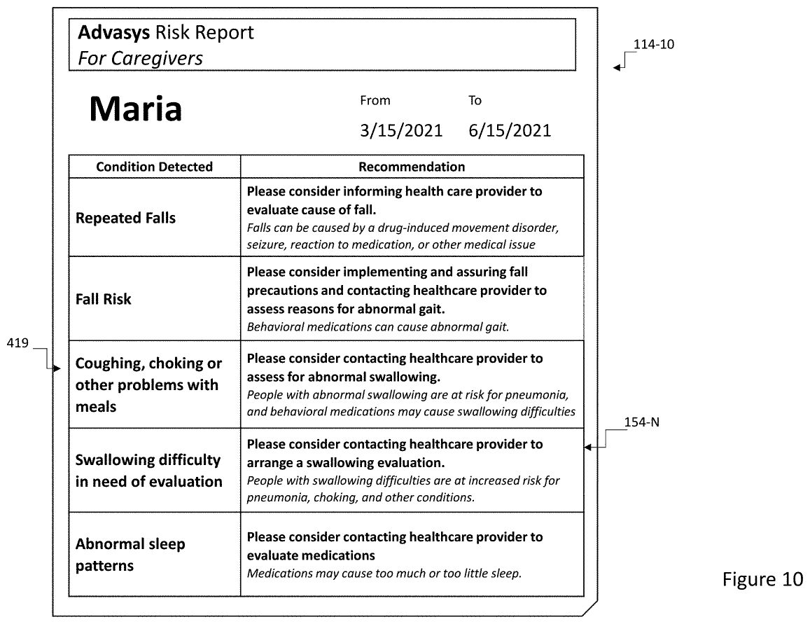

FIG. 10 is an example of a patient risk report for a caregiver generated as in FIGS. 2, 4 and 7. Caregivers may not need or appreciate the depth of information on the medical professional's report; hence a report 114-10 includes only the conditions 419 and provider recommendations that the patient or caregiver may have control over and are advised to know about.

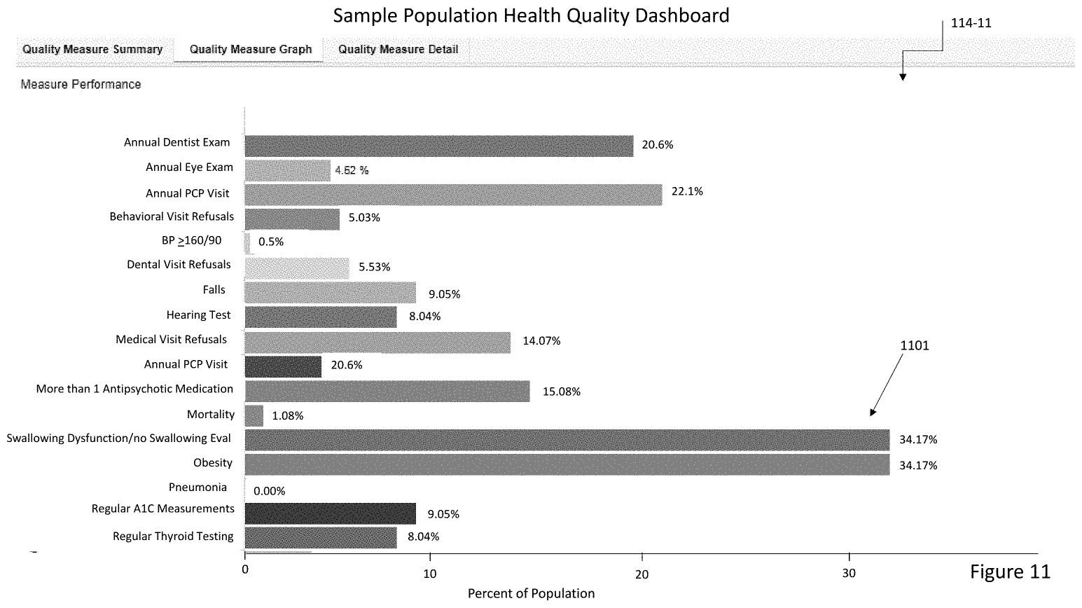

FIG. 11 is a population health quality report generated as in FIG. 2. The population health quality report 114-11 differs slightly in that it does not call out a particular patient, but rather presents statistical information across a patient group. FIG. 11 indicates a report applicable to a range of patients, thus amenable to a case worker or administration role. Individuals affected by each quality indicator (those in the numerator, with a defined population in the denominator) can be directly identified by clicking on the relevant bar in the graph for further expansion to the individual patients that comprise the group. With respect to FIGS. 9 and 10, patient Maria is be counted in the numerator of those who have swallowing difficulties or pneumonia and no history of swallowing evaluation, shown by arrow 1101. This assists in organizational or state health quality improvement initiatives and/or reporting mandates, for example.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for detecting and tracking health anomalies in patients having developmental and intellectual disabilities (D/ID) or dementia, comprising:
rendering an interface having a sequence of questions, each question pertaining to a symptom, each symptom based on an aspect of health and wellbeing known to be compromised in a patient having a self expression degradation due to mental impairment;
receiving a response for each symptom;
coalescing the received responses with a set of symptoms for determining a match between the received responses and combinations of symptoms indicative of a condition; and
rendering a provider recommendation corresponding to the condition on an output device.

2. The method of claim 1 wherein the sequence of questions are further subdivided into cognizance areas, each cognizance area define a grouping of symptoms pertaining to a common health topic.

3. The method of claim 2 wherein the cognizance areas include ADLs (Activities of Daily Living), observations, ambulation, communications, eating, elimination, environment, negative occurrences, stress, sleep, physiologic data from wearable sensor devices, substances, medications, and health problem list.

4. The method of claim 1 further comprising ordering each of the symptoms according to a symptom name and range applicable values for storage as responses to the symptom.

5. The method of claim 1 further comprising identifying a set of symptoms in a medical logic expression based on the likelihood that the identified symptoms are deterministic of the condition.

6. The method of claim 1 wherein coalescing further comprises:
identifying medical logic associated with one or more of the symptoms, the medical logic including an expression for computing a condition based on a value of one or more of the symptoms.
evaluating the medical logic for computing whether the condition is present; and
identifying a provider recommendation corresponding to the condition.

7. The method of claim 6 further comprising:
receiving a script based on medical expertise, the script defining a logical expression for concluding a condition based on an absence or presence of one or more symptoms stored and attributed to a patient; and
parsing the script by referencing, for a patient, the symptoms called for by the logical expression; and
computing an existence of the condition in the patient based on the parsing.

8. The method of claim 6 further comprising:
identifying an EHR (Electronic Healthcare Record) including medical records of the patient;
identifying a script syntax recognized for access to a repository under the control of the EHR;
determining a condition for which diagnoses is sought; and
defining, in the syntax, the medical logic for determining the presence of the condition in the patient based on the symptoms.

9. The method of claim 6 further comprising:
storing, in an EHR, an indication of each of the symptoms and a corresponding value pertaining to a patient;
storing, in the EHR, a medical record of the patient, the medical records including histories of at least one of procedures, medications and physiology;
executing a script, the script defining medical logic, the medical logic including syntax elements referencing the symptoms and at least one of the procedures, medications and physiology stored in the EHR medical record corresponding to the patient.

10. The method of claim 6 further comprising
identifying a type of a report for generation, the type based on a healthcare role of a requestor of the report;
mapping the condition to a provider recommendation based on the type of the report; and
rendering a report including the mapped provider recommendation in conjunction with rendering the condition.

11. The method of claim 5 wherein coalescing includes:
assigning a condition index based on the received responses to the symptoms;
storing the received responses in a repository;
identifying the condition index based on the medical logic expression; and
retrieving the provider recommendation and rendering to a second healthcare provider upon a successive visit.

12. A network communication and reporting device for detecting and tracking health anomalies in patients having developmental and intellectual disabilities (D/D) or dementia, comprising:
an interface for receiving responses to a sequence of questions, each question pertaining to a symptom, each symptom based on an aspect of health and wellbeing known to be compromised in a patient having a self expression degradation due to mental impairment;
a storage repository for storing a received response for each symptom;
an execution engine for coalescing the received responses with a set of conditions for determining a match between the received responses and combinations of symptoms indicative of a provider recommendation; and
an output device for rendering the matched provider recommendation.

13. The device of claim 12 further comprising a table for storing and ordering each of the symptoms according to a symptom name and range applicable values for storage as responses to the symptom.

14. The device of claim 12 further comprising a medical logic expression having a set of symptoms for determining if the identified conditions are deterministic of the condition.

15. The device of claim 12 wherein the execution engine is configured to:
identify medical logic associated with one or more of the symptoms, the medical logic including an expression for computing a condition based on a value of one or more of the symptoms.
evaluate the medical logic for computing whether the condition is present; and
identify a provider recommendation corresponding to the condition.

16. The device of claim 14 further comprising:
a table for storing a script based on medical expertise, the script defining a logical expression for concluding a condition based on an absence or presence of one or more symptoms stored and attributed to a patient; and
a parser for parsing the script by referencing, for a patient, the symptoms called for by the logical expression; and
the execution engine further configured to compute, based on the parsing, an existence of the condition in the patient.

17. The device of claim 14 further comprising:
a database having an EHR (Electronic Healthcare Record) including medical records of the patient;
a script syntax recognized for access to a repository under the control of the EHR for determining a condition for which diagnoses is sought; and
medical logic, defined in the syntax, for determining the presence of the condition in the patient based on the symptoms.

18. The method of claim 1 further comprising:
identifying a provider role based on an intended recipient of a report; and
mapping the condition to a provider recommendation based on the provider role.

19. A computer program embodying program code on a non-transitory medium that, when executed by a processor, performs steps for implementing a method for detecting and tracking health anomalies in patients having developmental and intellectual disabilities (D/ID) or dementia, the method comprising:
rendering an interface having a sequence of questions, each question pertaining to a symptom, each symptom based on an aspect of health and wellbeing known to be compromised in a patient having a self expression degradation due to mental impairment;
receiving a response for each symptom;
coalescing the received responses with a set of conditions for determining a match between the received responses and combinations of symptoms indicative of a provider recommendation; and
rendering the matched provider recommendation on an output device.

20. A system for detecting and tracking health anomalies in patients having developmental and intellectual disabilities (D/D) or dementia, the method comprising:
a user interface for rendering a sequence of questions, each question pertaining to a symptom, each symptom based on an aspect of health and wellbeing known to be compromised in a patient having a self expression degradation due to mental impairment;
an encounter form for receiving a response for each symptom;
a database for storing the symptoms and a set of conditions resulting from one or more of the symptoms;
medical logic for coalescing the received responses with a set of conditions for determining a match between the received responses and combinations of symptoms indicative of a provider recommendation based on a healthcare role; and
a report module for rendering the matched provider recommendation on an output device.
